(19) United States
(12) Patent Application Publication
(10) Pub. No.: US 2025/0025346 A1
(43) Pub. Date: Jan. 23, 2025

(54) LASER PROTECTION DEVICE, IN PARTICULAR LASER PROTECTION HELMET

(71) Applicant: Optrel Holding AG, Wattwil (CH)

(72) Inventors: Simon WIRZ, Niederteufen (CH); Martin ESPOSITO, Rapperswil (CH); Jonathan HEUSSER, Uerikon (CH)

(21) Appl. No.: 18/758,502

(22) Filed: Jun. 28, 2024

(30) Foreign Application Priority Data
Jul. 19, 2023 (EP) 23186438.0

(51) Int. Cl. A61F 9/06

(52) U.S. Cl. CPC A61F 9/065; A61F 9/061

(57) ABSTRACT

A laser protection device, in particular a laser protection helmet, includes at least one optical glare protection filter, which in particular has a nose cutout, further includes at least one sensor unit which is configured for a detection of a working state and for actuating, as a function thereof, a transmittance of the glare protection filter, and includes at least one shield unit which is configured to protect a face of a user and in which the at least one glare protection filter is fixedly accommodated, wherein the laser protection device further comprises at least one radiation protection unit, which extends around the glare protection filter and at least partially consists of a laser-absorbing, in particular metallic, material.

### CROSS-REFERENCE TO RELATED APPLICATION

This U.S. patent application is based on and incorporates herein by reference European patent application EP 23186438.0, filed on Jul. 19, 2023.

### Prior Art

The invention concerns a laser protection device, in particular a laser protection helmet.

A laser protection device, in particular a laser protection helmet, with at least one optical glare protection filter, which in particular has a nose cutout, with at least one sensor unit configured for a detection of a working state and for controlling, as a function thereof, a transmittance of the glare protection, with at least one shield unit which is configured to protect a face of an operator and in which the at least one glare protection filter is fixedly accommodated, has already been proposed.

The objective of the invention is in particular to provide a generic device with improved properties regarding protection and/or comfort. The objective is achieved according to the invention.

### Advantages of the Invention

The invention is based on a laser protection device, in particular a laser protection helmet, with at least one optical glare protection filter, which in particular has a nose cutout, with at least one sensor unit which is configured for a detection of a working state and for controlling, as a function thereof, a transmittance of the glare protection filter, with at least one shield unit which is configured to protect a face of an operator and in which the at least one glare protection filter is fixedly accommodated.

It is proposed that the laser protection device comprises a radiation protection unit, which extends around the glare protection filter and at least partially consists of a laser- absorbing, in particular metallic, material.

A “laser protection device” is in particular to mean, in this context, a device which is configured for a protection of a user against excessive brightness and/or sparks and/or a laser beam, in particular a laser welding beam. Preferably, this is in particular to mean a device used for protecting eyes and/or a face region of a user during a welding and/or grinding process. Preferably, this is in particular to mean a glare protection device which in particular serves for protecting a user's eyes at least during a welding process, in particular a laser welding process. A variety of implementations of a laser protection device are conceivable which are deemed expedient by someone skilled in the art, such as for example as a laser welding helmet, as a laser welding screen, as a laser welding mask and/or as a laser welding shield. Furthermore, in this context, an “optical glare protection filter” is in particular to mean an optical filter which in particular forms a protective glass and/or a protective plastic glass. Preferably, this is in particular to mean an optical filter whose light transmittance is realized so as to be adjustable. Preferentially, this is in particular to mean an optical welding protection filter, in particular a laser welding protection filter, with automatic darkening. Particularly preferably, the glare protection filter comprises at least one liquid-crystal plane which is switchable in transmittance. A variety of implementations of the optical glare protection filter are conceivable which are deemed expedient by someone skilled in the art, but in particular this is to mean an ADF, also called an “automatic darkening filter” or an “automatic welder protection filter”.

Furthermore, a “nose cutout” is in particular to mean, in this context, an immaterial recess in an at least partially translucent subregion of the glare protection filter, which is in at least one operative position of the laser protection device at least partially configured for at least partially accommodating a user's nose. Preferably, the cutout is surrounded in each point in at least one plane, in particular parallel to a main extension plane of the glare protection filter, in an angle range of at least 180°, by a material subregion, in particular an at least partially translucent subregion, of the glare protection filter.

Preferably, in at least one operative position of the laser protection device, the glare protection filter engages around a user's nose. Particularly preferentially, a vertical extent of the nose cutout is at least 10%, preferably at least 30%, preferably at least 50% and particularly preferably at least 55% of a vertical extent of the glare protection filter. By a “sensor unit” is in particular, in this context, a unit to be understood which is configured for recording at least one characteristic variable and/or a physical property, wherein the recording may take place actively, like in particular by generating and emitting an electrical measurement signal, and/or passively, like in particular by a detection of property changes of a sensor component. A variety of sensor units deemed expedient by someone skilled in the art are conceivable. Preferably, the sensor unit in particular comprises at least one photocell. Preferably the photocell is in particular configured at least for an optical detection of an electric arc. Furthermore, in this context, a “shield unit” is in particular to mean a unit which, in a regular operative position, is arranged in front of a user's face. Preferably, this is in particular to mean a unit which, in an operative position of the laser protection device, in particular covers at least a substantial portion of a user's face. Preferably this is in particular to mean a unit configured for a protection of a face, like for example against flying sparks. Preferably the shield unit is in particular configured to form a protective barrier between a work area and the user's face. “Configured” is in particular to mean specifically programmed, designed and/or equipped. By an object being configured for a specific function, it is in particular to be understood that the object fulfils and/or performs this specific function in at least one application state and/or operation state.

By a “radiation protection unit” is in particular, in this context, a unit to be understood which is configured for a protection of a user's eyes against radiation, in particular laser radiation, which is in particular directed past the optical glare protection filter to at least one eye of the user. Preferably the radiation protection unit is configured to protect a peripheral field of view and/or a face of a user against laser radiation. The radiation protection unit is in particular configured to shield the user's eyes in a region around the optical glare protection filter. The radiation protection unit is in particular configured to be impenetrable for laser radiation. In contrast, the laser radiation is in particular capable of penetrating the shield unit. A “laser-absorbing material” is in particular to mean, in this context, a material which absorbs and/or reflects laser radiation at least in a defined wavelength range, in particular completely. Herein different materials are conceivable which are deemed expedient by someone skilled in the art, such as for example metallic materials, in particular aluminum and/or aluminum alloys. Preferably the laser-absorbing material is at least substantially, in particular completely, impenetrable for laser radiation.

The laser protection device according to the invention in particular enables advantageous worker protection during the lasering process, in particular during a laser welding process. In particular, damage to a user's eyes by an incorrectly directed laser beam can be avoided. At the same time, in particular a mechanical protection of a user's face, for example against flying parts or sparks, may also be enabled. Furthermore, an advantageously high level of operating and/or wearing comfort is enabled.

Furthermore, it is proposed that the radiation protection unit comprises at least one radiation protection wall, which extends around the glare protection filter and consists of a laser-absorbing, in particular metallic, material. The radiation protection wall is in particular formed as a shaped sheet metal part and/or as a cast part, which extends around the optical glare protection filter at a peripheral edge of the optical glare protection filter. Preferably the radiation protection wall directly adjoins the glare protection filter. The radiation protection wall may in particular consist completely of metal or, for example, of a composite material. Preferably the radiation protection wall is in particular embodied as a narrow circumferential web which, in a worn state, is configured to extend towards a user's face. This in particular allows providing an advantageously safe and lightweight radiation protection unit.

Furthermore, it is proposed that the at least one radiation protection wall consists of aluminum and/or of an aluminum alloy. Preferably the radiation protection wall consists completely of aluminum or of an aluminum alloy. However, other materials are also conceivable which are deemed expedient by someone skilled in the art. This in particular allows providing an advantageously safe and lightweight radiation protection unit.

It is further proposed that the at least one radiation protection wall projects at least substantially perpendicularly to a main extension plane of the glare protection filter into an interior space of the shield unit. Preferably, in a worn state, the at least one radiation protection wall projects, at least substantially perpendicularly to a main extension plane of the glare protection filter, into an interior space of the shield unit towards a user's face. The term “substantially perpendicularly” is here in particular meant to define an orientation of a direction relative to a reference direction, wherein the direction and the reference direction, in particular when viewed in a projection plane, include an angle of 90° and the angle has a maximum deviation of in particular less than 16°, advantageously less than 12°, preferably less than 8° and particularly advantageously less than 2°. By a “main extension plane” of a structural unit is in particular a plane to be understood which is parallel to a largest side face of a smallest imaginary cuboid just still completely enclosing the structural unit, and in particular runs through the center point of the cuboid. The radiation protection wall in particular forms a nose cutout, in which the radiation protection wall has a reduced height. Next to the nose cutout, the radiation protection wall in particular has an at least approximately constant height. This in particular allows providing advantageously safe shielding. In particular, an advantageously safe radiation protection unit can be provided. Furthermore, an advantageously high wearing comfort may be provided.

Beyond this it is proposed that the at least one radiation protection unit comprises at least one shield insert, which consists of a laser-absorbing, in particular metallic, material. Preferably, the shield insert consists at least largely of aluminum and/or of an aluminum alloy. The shield insert is in particular directly coupled with the shield unit and reinforces the shield unit. Preferably the shield insert region-wise forms a two-walled design of the shield unit. Preferentially the shield insert adjoins the shield unit on an inner side of the shield unit, wherein the shield insert in particular follows a shape of the shield unit. Furthermore, it is proposed that the shield insert is arranged in a mouth region of a user behind the shield unit. The shield unit in particular comprises a forehead region, a chin region, and an eye region that is arranged between the forehead region and the chin region. The forehead region, the chin region and the eye region are preferably realized integrally. Preferably the forehead region and the chin region are shaped in substantially convex fashion on an outer side facing away from the user's face. The shield insert is in particular arranged in the chin region of the shield unit. This in particular allows providing advantageously safe shielding. In particular, an advantageously safe radiation protection unit can be provided. Furthermore, an advantageously high level of wearing comfort may be provided.

It is further proposed that the radiation protection unit is different from the shield unit. The radiation protection unit is in particular implemented by a separate component. The radiation protection unit is in particular connected to the shield unit. The radiation protection unit is in particular connected to the shield unit via a protective cassette. The shield unit in particular consists of a material different from the radiation protection unit. The shield unit in particular consists at least partially of plastic. Preferably the shield unit consists completely of plastic. The radiation protection unit is in particular detachably connected to the shield unit. This in particular allows providing an advantageously modular design of the laser protection device. In particular, an advantageous protection of the user is enabled.

Furthermore, it is proposed that the laser protection device comprises a head fastening unit for a fastening to a head of a user, and comprises an adjustment unit by means of which a position of the at least one shield unit is realized so as to be movable in a defined manner relative to the at least one head fastening unit. In this context, a “head fastening unit” is in particular to mean a unit which is configured for a fastening, in particular a fixing, of the laser protection device to the head of the user. For this purpose, the unit is preferably configured to at least partially engages around a user's head. In this context, “at least partially engage around” is in particular to mean that a head is encompassed by the head fastening unit in at least one plane in an angle range of at least 160°, preferably of at least 180°, preferentially of at least 270° and particularly preferentially of at least 360°.

Particularly preferably, the head fastening unit is in a regular operation state configured to rest against at least a forehead and against a back of a user's head. Furthermore, in this context, an “adjustment unit” is in particular to mean a unit by means of which, in particular manually, a position of the at least one shield unit is realized so as to be adjustable in a defined manner relative to the at least one head fastening unit. Preferably the adjustment unit comprises at least one adjustment member via which a user may change a position and/or a situation of the shield unit in a defined manner relative to the at least one head fastening unit. Particularly preferably, the adjustment unit is in particular configured at least for guiding a movement of the at least one shield unit in a defined manner relative to the at least one head fastening unit. In principle, it is also conceivable that the shield unit is moved directly relative to the head fastening unit, wherein a movement is in this case guided in a defined manner in particular by the adjustment unit. By “defined” is in particular to be understood that a movement of the shield unit is guided. Preferably, this is also to mean that a limit of a movement of the shield unit relative to the at least one head fastening unit is predetermined by the adjustment unit. Particularly preferably, this is in particular to mean that, in particular within the limits of its movability, the shield unit is fixable, in particular lockable, in a plurality of positions relative to the at least one head fastening unit. This in particular allows achieving an advantageously variable adjustment of a position of the shield unit. In particular, this allows the shield unit being optimally positioned in a user-specific manner. As a result, the optical glare protection filter may preferably be realized so as to be advantageously small and/or a field of view may be realized so as to be advantageously large.

Furthermore, it is proposed that a direct distance between the eyes of the user and the optical glare protection filter is realized so as to be adjustable by means of the at least one adjustment unit. Preferably, a position of the at least one shield unit is realized so as to be fixable in at least two operative positions relative to the at least one head fastening unit by means of the at least one adjustment unit. An “operative position” is in particular to mean, in this context, a position of the shield unit in which the shield unit performs and/or can perform a designated protection function. Preferably, this is in particular to mean a position in which the shield unit is arranged in front of a user's face. Particularly preferably, the at least two fixable operative positions differ in particular by a distance between the optical glare protection filter and the user's eyes and/or in an angle between the shield unit and the head fastening unit and/or in a height at which the optical glare protection filter is arranged relative to the user's head. Preferably, a distance between the user's eyes and the optical glare protection filter is adjustable during operation perpendicularly to a head axis by means of the at least one adjustment unit. Preferentially, a distance between the user's eyes and the optical glare protection filter is adjustable during operation approximately perpendicularly to a main extension plane of the glare protection filter by means of the at least one adjustment unit. A “direct distance” is in particular to mean, in this context, a distance between the user's eyes and the optical glare protection filter during operation, that is in the case of direct eye contact. This in particular allows achieving an advantageously variable adjustment of a position of the shield unit. Preferably this allows the shield unit being optimally positioned in a user-specific manner. In this way the longitudinal position of the optical glare protection filter can advantageously be adjusted depending on a welder's head circumference and well-being. Furthermore, this allows optimal adaption of a longitudinal position of the optical glare protection filter for wearers of spectacles. The closer to the face the optical glare protection filter is arranged, the greater the field of view will be for the user.

It is also proposed that the at least one radiation protection unit comprises at least one face stop, which in an end position of the radiation protection unit is configured to contact a face of the user. By a “face stop” is in particular, in this context, a stop to be understood which is configured to contact a user's face for defining the end position of the radiation protection unit relative to the user's face. Preferably, this is in particular to mean a stop whose end position depends on a shape of the user's face. Particularly preferably, this is in particular to mean a stop wherein a stop position is defined by a contact between face and face stop. This allows a reliable definition of an end position of the radiation protection unit. Furthermore, in this way the radiation protection unit can be reliably brought into an operative position. In particular, this allows achieving optimal positioning of the radiation protection unit relative to the user's face. This in turn allows achieving optimal shielding. Preferably, a face-specific stop may be provided. Preferably, the at least one face stop is formed by a nose rest, which in at least one operative position is configured for contacting a user's nose. Preferably, in at least one operative position, the nose rest is configured for contacting a user's bridge of the nose. Preferentially, in at least one operative position, the nose rest is configured for contacting a nose pyramid of a user's nose. Particularly preferably, the nose rest is formed by a spectacle-like nose rest. Preferably, the nose rest comprises at least one nose pad. Preferentially, the nose rest comprises at least two nose pads. Particularly preferably, a position and/or a situation of the nose pads are/is adjustable. The nose rest is in particular adaptable to a shape of a nose. This allows a reliable definition of an end position of the shield unit. In particular, this allows achieving optimal positioning of the radiation protection unit relative to a user's face. Preferably a position of the shield unit may be oriented with respect to a center of the face. A reliable positioning of the radiation protection unit relative to the user's eyes is enabled. Moreover, at least a small portion of a weight of the laser protection device can be supported on the nose. This in particular allows avoiding a downward sliding of the laser protection device during operation.

Furthermore, the invention is based on the radiation protection unit of the laser protection device. The radiation protection unit may in particular be connected to the shield unit of the laser protection device in an exchangeable fashion.

The laser protection device according to the invention shall not be limited to the above- described application and implementation. In particular, in order to fulfil a functionality that is described here, the laser protection device according to the invention may have a number of individual elements, components and units differing from a number given here.

### DRAWINGS

Further advantages will become apparent from the following description of the drawings. In the drawings an embodiment of the invention is illustrated. The drawings, the description and the claims contain numerous features in combination. Someone skilled in the art will purposefully also consider the features individually and will find further expedient combinations.

In the drawings:

FIG. 1 shows the laser protection device according to the invention with a shield unit, with a head fastening unit, with an optical glare protection filter and with an attachment panel on a user's head, in a schematic illustration,

FIG. 2 shows the laser protection device according to the invention with the shield unit, with the head fastening unit, with the optical glare protection filter and with a radiation protection unit, in a schematic rear view,

FIG. 3 shows the radiation protection unit of the laser protection device according to the invention in a schematic illustration,

FIG. 4 shows the radiation protection unit of the laser protection device according to the invention in a further schematic illustration, and

FIG. 5 shows a schematic sectional view of the laser protection device with the optical glare protection filter in a state when worn.

### DESCRIPTION OF THE EXEMPLARY EMBODIMENT

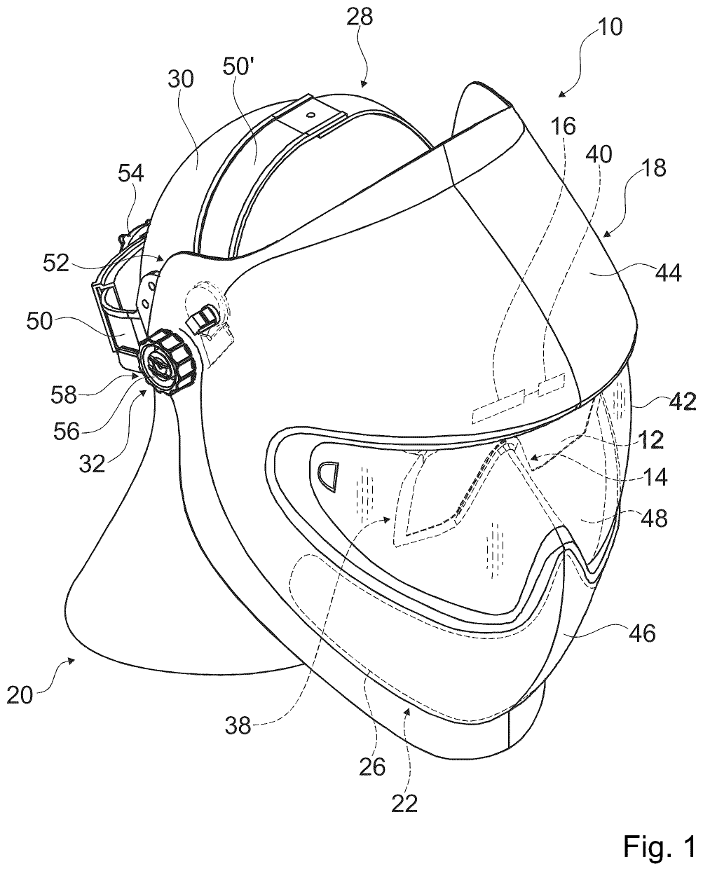

FIG. 1 shows a laser protection device 10 on a head 30 of a user 20. The laser protection device 10 is configured to be worn on the head 30 by a user 20 during operation. FIG. 1 shows the laser protection device 10 in an operative position. The laser protection device 10 is realized as a laser welding helmet. In principle, however, another implementation of the laser protection device 10, deemed expedient by someone skilled in the art, would also be conceivable.

The laser protection device 10 comprises an optical glare protection filter 12. Furthermore, the laser protection device 10 comprises a sensor unit 16. The sensor unit 16 is configured for a detection of a working state and for actuating, as a function thereof, a transmittance of the glare protection filter 12. The sensor unit 16 comprises at least one sensor configured to detect a welding process or the occurrence of a bright light, which could damage or otherwise affect the eyes 34 of a user 20. The sensor of the sensor unit 16 is embodied as a photocell. In principle, however, another implementation of the sensor of the sensor unit 16, deemed expedient by someone skilled in the art, would also be conceivable. Furthermore, the laser protection device 10 comprises a protective cassette 38. The optical glare protection filter 12 and the sensor unit 16 form part of the protective cassette 38. Furthermore, the protective cassette 38 comprises an electronics unit 40. The electronics unit 40 is coupled with the sensor unit 16. The electronics unit 40 is configured for processing the data of the sensor unit 16 and for actuating the glare protection filter 12 as a function thereof. The electronics unit 40 comprises a battery and/or solar cell, not visible in detail, for a power supply.

The optical glare protection filter 12 is embodied as an electro-optical filter. The optical glare protection filter 12 is realized as an automatic darkening filter, ADF for short. The optical glare protection filter 12 consists of several layers. The optical glare protection filter 12 is realized as a multilayer composite. A number of layers is herein merely exemplary and may vary in principle.

The glare protection filter 12 has a rectangular basic shape. A vertical extent of the glare protection filter 12 is herein, in a regular operation state, smaller than a horizontal extent of the glare protection filter 12. The glare protection filter 12 further comprises a nose cutout 14. The nose cutout 14 is formed by an immaterial recess in a material part of the glare protection filter 12. The nose cutout 14 is formed by an immaterial recess in a material, at least partially translucent subregion of the glare protection filter 12. In an operative position of the laser protection device 10, the nose cutout 14 is configured to partially accommodate the nose of the user 20. In an operative position, the glare protection filter 12 partially engages around the nose of the user 20 in the region of the nose cutout 14. The glare protection filter 12 has a substantially rectangular basic shape, wherein the nose cutout 14 projects into the rectangular basic shape. The nose cutout 14 has a substantially triangular shape. The two sides delimited by the material part of the glare protection filter 12 have an angle of approximately 54° to each other.

Furthermore, the laser protection device 10 comprises a shield unit 18. The glare protection filter 12 is fixedly accommodated in the shield unit 18. The glare protection filter 12 is accommodated in the shield unit 18 in a positionally fixed manner. The glare protection filter 12 is fitted in a recess in the shield unit 18. The entire protective cassette 38 is fitted in a recess in the shield unit 18. The shield unit 18 consists of a substantially dimensionally stable material. The shield unit 18 consists of a plastic, which is in particular resistant to sparks and/or other influences that occur during welding. The shield unit 18 is configured to cover and protect a face and/or the head 30 of the user 20, in particular in accordance with the relevant standards for welding masks. The shield unit 18 has a shape that is partially adapted to a head shape. In a worn state of the laser protection device 10, the shield unit 18 is partially bent around the face of a user 20. In a worn state of the laser protection device 10, the shield unit 18 partially accommodates the face of a user 20. The shield unit 18 comprises a forehead region 44, a chin region 46, and an eye region 48 that is arranged between the forehead region 44 and the chin region 46. The forehead region 44, the chin region 46 and the eye region 48 are realized integrally. The forehead region 44 and the chin region 46 are shaped in substantially convex fashion on an outer side that faces away from the face of the user 20. The eye region 48 comprises a receptacle for receiving the protective cassette 38. The eye region 48 is depressed towards the receptacle of the protective cassette 38. The receptacle for the protective cassette 38 is arranged offset inwards with respect to a basic shape of the shield unit 18, therefore towards the face of the user 20. The receptacle for the protective cassette 38 is arranged offset inwards with respect to the forehead region 44 and with respect to the chin region 46.

The laser protection device 10 furthermore comprises an attachment panel 42. The attachment panel 42 is connected to the shield unit 18 via latching elements which are not visible in detail. Preferably, the attachment panel 42 comprises two opposite-situated latching recesses, in each of which a latching element of the shield unit 18 engages in a latching manner. Due to the latching, the attachment panel 42 can be easily disassembled. This allows simple cleaning and/or simple exchange. The attachment panel 42 is realized in transparent fashion. The attachment panel 42 is configured for a protection of the protective cassette 38. The attachment panel 42 covers the protective cassette 38 from the outside. This allows avoiding damage to the protective cassette 38. If the attachment panel 42 is damaged, it is simply and cost-effectively replaceable, in particular in contrast to the protective cassette 38.

Furthermore, the laser protection device 10 comprises a head fastening unit 28. The head fastening unit 28 is configured for a fastening to the head 30 of the user 20. The head fastening unit 28 is realized as a head strap. The head fastening unit 28 comprises several head strap elements 50, 50′, which are in each case configured to extend at least partly around the head 30 of the user 20. The head fastening unit 28 comprises three head strap elements 50, 50′, which are respectively configured to extend in different planes around the head 30 of the user 20. The three head strap elements 50, 50′ are realized in strap-shaped fashion. A first head strap element 50 extends from a first common connection point 52 above an ear of the user 20 around a back of the head 30 of the user 20 to a second common connection point above the other ear of the user 20. An effective length of the first head strap element 50 is adjustable. The head fastening unit 28 comprises an adjustment element 54 on the head strap element 50. The adjustment element 54 is realized as an adjustment wheel, which is known for example from helmets and by means of which an effective length of the first head strap element 50 can be adjusted by rotation. A second head strap element 50′ extends from the first common connection point 52 over an upper head of the head 30 of the user 20 to the second common connection point above the other ear of the user 20. The second head strap element 50′ extends from the first common connection point 52 approximately at an angle of 120° with respect to the first head strap element 50. An effective length of the second head strap element 50′ is adjustable. For this purpose, the second head strap element 50′ is realized in two parts. The two parts of the second head strap element 50′ are connected via an adjustable closure unit. The adjustable closure unit is realized as a snap-back closure. A third head strap element extends from the first common connection point 52 around a forehead of the head 30 of the user 20 to the second common connection point above the other ear of the user 20. The third head strap element extends from the first common connection point 52 approximately at an angle of 90° with respect to the second head strap element 50′. The three head strap elements 50, 50′ of the head fastening unit 28 are fixedly connected to one another via the connection points 52. The head fastening unit 28 consists of a partially elastic plastic. In principle, however, another material, deemed expedient by someone skilled in the art, would also be conceivable.

Beyond this the laser protection device 10 comprises an adjustment unit 32. A position of the shield unit 18 is realized so as to be movable in a defined manner relative to the head fastening unit 28 by means of the adjustment unit 32. By means of the adjustment unit 32, a position of the shield unit 18 can be adjusted in a defined manner relative to the head fastening unit 28. A position of the shield unit 18 is realized so as to be fixable in at least two operative positions relative to the at least one head fastening unit 28 by means of the adjustment unit 32. A position of the shield unit 18 is realized so as to be fixable in a delimited region in any operative position relative to the at least one head fastening unit 28 by means of the adjustment unit 32. By means of the adjustment unit 32, a situation of the shield unit 18 in an operative position can be freely selected, wherein the situation of the shield unit 18 can be fixed. The shield unit 18 is connected to the head fastening unit 28 via the adjustment unit 32. The adjustment unit 32 is connected to the head fastening unit 28 in the two connection points 52, 52′. Furthermore, the adjustment unit 32 is connected to the shield unit 18 in a peripheral region of the forehead region 44 of the shield unit 18. The adjustment unit 32 comprises a base element. The base element is fixedly connected to the head fastening unit 28 in the first connection point 52 of the head fastening unit 28. The adjustment unit 32 further comprises a second base element, which is mirror-symmetrical to the base element. The second base element is fixedly connected to the head fastening unit 28 in the second connection point of the head fastening unit 28.

The adjustment unit 32 further comprises a rotary wheel 56, which is rotatably supported on the shield unit 18. Furthermore, the rotary wheel 56 is translationally guided in a guide rail of the first base element. The rotary wheel 56 is guided in the guide rail of the first base element perpendicularly to the head axis. A direct distance between the eyes 34 of the user 20 and the optical glare protection filter 12 is realized so as to be adjustable by means of the adjustment unit 32. A direct distance d1, d2 between the eyes 34 of the user 20 and the optical glare protection filter 12 can be adjusted in the horizontal direction, i.e. perpendicularly to the head axis, by means of the adjustment unit 32. The direct distance d1, d2 between the eyes 34 of the user 20 and the optical glare protection filter 12 can be changed by means of the rotary wheels 56 of the adjustment unit 32. A position of the shield unit 18 in a longitudinal direction relative to the face of the user 20 can be changed by means of the rotary wheels 56. Once a desired longitudinal position has been found, the position can be fixed by rotating the rotary wheels 56. Moreover, in an operative position an angle of the shield unit 18 relative to the head fastening unit 28 is realized so as to be adjustable by means of the adjustment unit 32. For this purpose the adjustment unit 32 comprises a lever 58.

The adjustment unit 32 is realized according to EP 3 213 726 A1. A description of the adjustment unit 32 of EP 3 213 726 A1 is therefore also applicable to the present description and is to be considered as part of the disclosure.

Furthermore, the laser protection device 10 comprises a radiation protection unit 22. The radiation protection unit 22 extends around the glare protection filter 12 and at least partially consists of a laser-absorbing, in particular metallic, material. The radiation protection unit 22 comprises a radiation protection wall 24, which extends around the glare protection filter 12 and consists of a metallic material. The radiation protection wall 24 consists of aluminum and/or of an aluminum alloy. The radiation protection wall 24 is made of a shaped sheet metal part which extends around the optical glare protection filter 12 at a peripheral edge of the optical glare protection filter 12. The radiation protection wall 24 extends completely circumferentially along a peripheral edge of the optical glare protection filter 12. The radiation protection wall 24 directly adjoins the glare protection filter 12. The radiation protection wall 24 directly adjoins a frame of the optical glare protection filter 12. The radiation protection wall 24 is embodied as a narrow circumferential web which, in a worn state, is configured to extend towards the face of a user 20. The radiation protection wall 24 comprises several subregions. The radiation protection wall 24 comprises a left-hand and a right-hand lateral wall portion 84, 84′, which in each case shield a field of view of the user 20 towards a side. The lateral wall portions 84, 84′ each have a rectangular basic shape. The lateral wall portions 84, 84′ in particular extend vertically. Furthermore, the radiation protection wall 24 comprises a frontal wall portion 86, which shields a field of view of the user 20 towards the top. The frontal wall portion 86 extends substantially along a forehead of the user 20. The frontal wall portion 86 in particular extends horizontally. The partial front wall 86 adjoins the lateral wall portions 84, 84′ substantially perpendicularly. The frontal wall portion 86 has a basic shape which is curved on one side. The radiation protection wall 24 further comprises a nose wall portion 88, which shields a field of view of the user 20 downwards. The nose wall portion 88 extends substantially along the cheeks and the nose of the user 20. The nose wall portion 88 follows the nose cutout 14. The nose wall portion 88 in particular extends horizontally. The nose wall portion 88 adjoins the lateral wall portions 84, 84′ substantially perpendicularly. The nose wall portion 88 has a basic shape which is curved on one side. A height of the web herein varies in such way that the radiation protection wall 24 at least partially follows a contour of the face. The radiation protection wall 24 in particular has a maximum height to the left and to the right of the lateral wall portions 84, 84′, while the height in a region of the frontal wall portion 86 is continuously reduced towards a center. Furthermore, the height of the radiation protection wall 24 in a region of the nose wall portion 88 is minimal, wherein the height of the radiation protection wall 24 continuously decreases towards a center of the nose cutout 14. The radiation protection wall 24 projects, at least substantially perpendicularly to a main extension plane of the glare protection filter 12, into an interior space of the shield unit 18. The height of the radiation protection wall 24 therefore relates in particular to a direction perpendicular to the main extension plane of the glare protection filter 12. In a worn state, the radiation protection wall 24 projects, at least substantially perpendicularly to a main extension plane of the glare protection filter 12, into an interior space of the shield unit 18 towards the face of a user 20. By way of example, the radiation protection wall 24 is screwed with the protective cassette 38. For this purpose, the radiation protection wall 24 has on both sides fastening tabs 80, 80′ through which screws 82, 82′ can be screwed for a connection with the protective cassette 38. However, other connection techniques, deemed expedient by someone skilled in the art, are also conceivable.

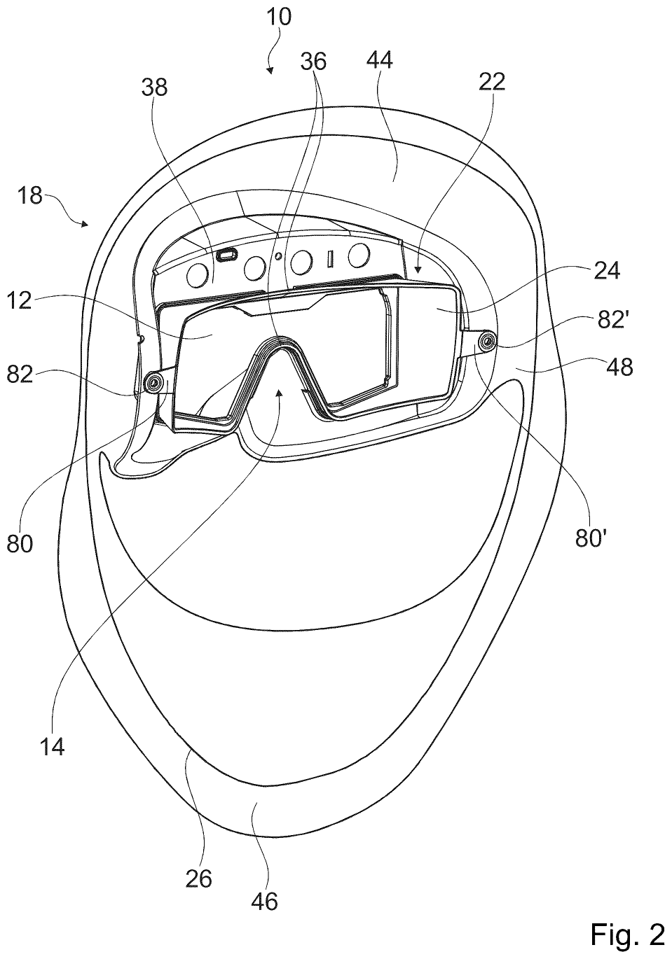

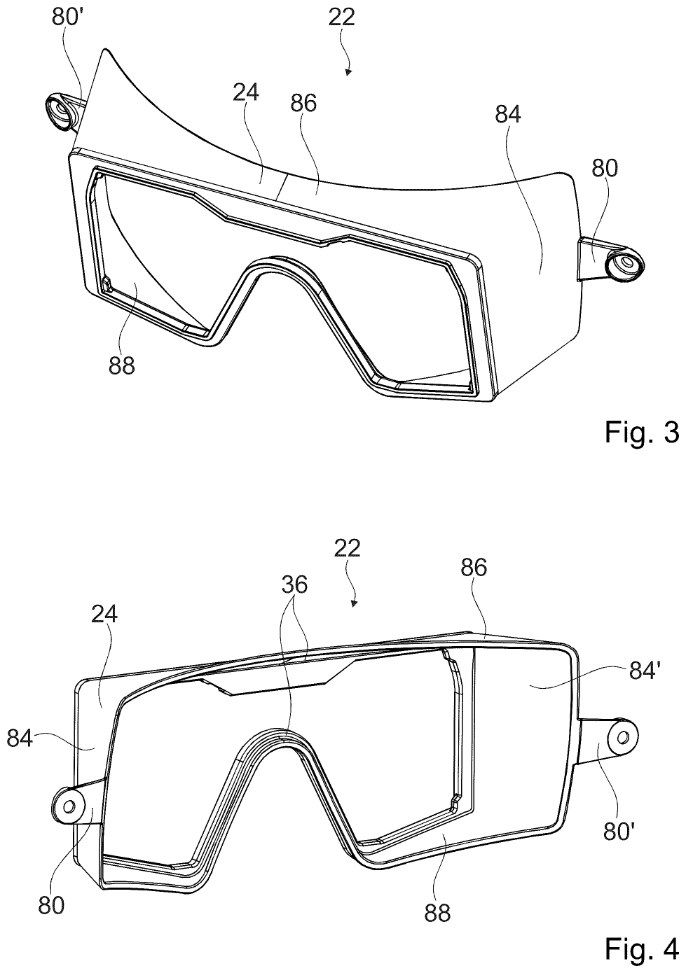

Furthermore, the radiation protection unit 22 comprises a shield insert 26, which consists of a laser-absorbing, in particular metallic, material. The shield insert 26 consists at least largely of aluminum and/or of an aluminum alloy. The shield insert 26 is directly coupled with the shield unit 18 and reinforces the shield unit. The shield insert 26 forms a region- wise two-walled design of the shield unit 18. The shield insert 26 adjoins the shield unit 18 on an inner side of the shield unit 18, wherein the shield insert 26 follows a shape of the shield unit 18. The shield insert 26 is arranged in a mouth region of a user 20 behind the shield unit 18. The shield insert 26 is arranged in the chin region 46 of the shield unit 18. The shield insert 26 extends over a large portion of the chin region 46 of the shield unit 18. The shield insert 26 extends at least partially into the nose cutout 14. The shield insert 26 is glued with the shield unit 18. However, other connection techniques deemed expedient by someone skilled in the art are also conceivable (FIGS. 2, 3, 4).

The radiation protection unit 22 is different from the shield unit 18. The radiation protection unit 22 is fixedly connected to the shield unit 18. The radiation protection unit 22 is fixedly connected to the shield unit 18 via the protective cassette 38.

The radiation protection unit 22 further comprises a face stop 36. In an end position of the radiation protection unit 22, the face stop 36 is configured to contact the face of a user 20. A stop position is in particular defined by a contact between face and face stop 36. A stop position herein defines an operative position. The face stop 36 of the radiation protection unit 22 is configured for a positioning of the radiation protection unit 22 relative to the face of a user 20. The face stop 36 is realized as a nose rest and/or forehead rest. The face stop 36 is exemplarily realized as a spectacle-like nose rest. In an operative position, the face stop 36 is configured for contacting a nose and/or a forehead of the user 20. In an operative position, the face stop 36 is configured for contacting a bridge of the nose of the user 20. This allows reliably defining an end position of the radiation protection unit 22. In particular, this allows achieving a reliable positioning of the radiation protection unit 22 relative to the eyes 34 of the user 20. The face stop 36 is arranged in a region of the nose cutout 14 of the optical glare protection filter 12. In principle, however, another implementation and/or arrangement of the face stop 36, deemed expedient by someone skilled in the art, would also be conceivable (FIG. 2).

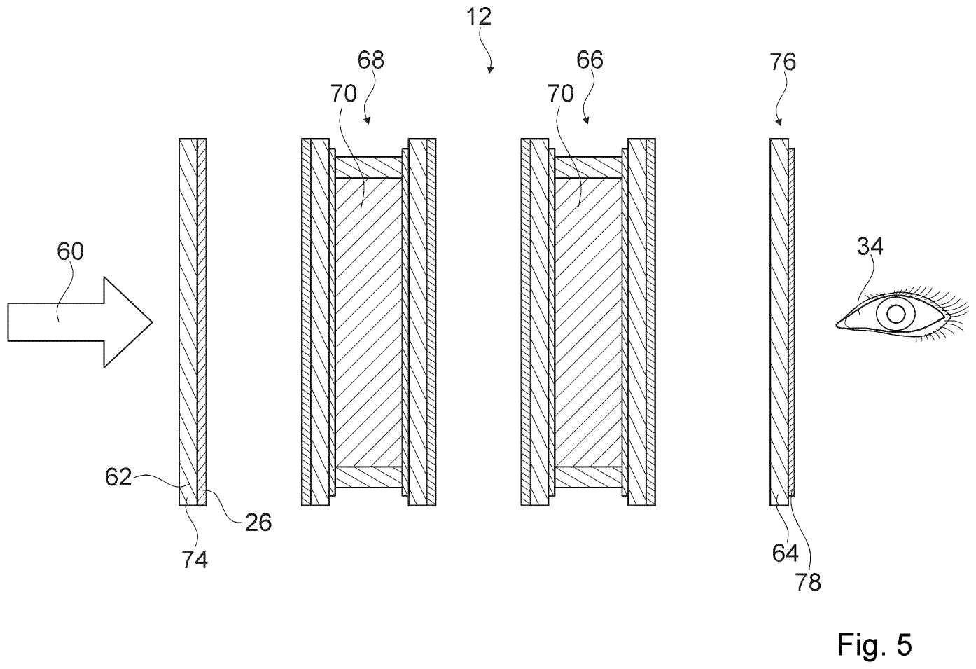

FIG. 5 shows a schematic side view of the optical glare protection filter 12. A typical incidence direction 60 for electromagnetic radiation that is to be shielded off is indicated in arrow direction. A position of a user is indicated by an eye 34 on a side of the optical glare protection filter 12 that is situated opposite the incidence direction 60. The optical glare protection filter 12 comprises a cover disk 62. The optical glare protection filter 12 comprises a further cover disk 64. Furthermore, the optical glare protection filter 12 comprises a liquid-crystal cell 66 and a further liquid-crystal cell 68. The liquid-crystal cell 66 and the further liquid-crystal cell 68 are arranged in the viewing direction between the cover disk 62 and the further cover disk 64. The cover disk 62 is arranged on a side of the optical glare protection filter 12 that faces away from the user 20 in the worn state. The further cover disk 64 is arranged on a side of the optical glare protection filter 12 that faces towards the user 20 in the worn state. The cover disk 62 and the further cover disk 64 are configured for a protection of the liquid-crystal cell 66 and the further liquid-crystal cell 68 and their components. The liquid-crystal cells 66, 68 in each case have an active layer 70. The active layer 70 is configured for manipulating a polarization direction of incident light via an actuation by the electronics unit 40. The active layer 70 is configured to rotate the polarization direction of light beams depending on an applied voltage. The active layer 70 is at least partially liquid. The active layer 70 is realized as a liquid-crystal layer.

The optical glare protection filter 12 comprises a passive filter unit 72. The passive filter unit 72 comprises an absorptive infrared filter element 74. The infrared filter element 74 is realized as an absorption glass. The infrared filter element 74 is realized integrally with the cover panel 62.

The optical glare protection filter 12 comprises an antireflection unit 76. The antireflection unit 76 has an antireflection coating 78. The antireflection coating 78 is arranged on a surface of the further cover disk 64. The antireflection coating 78 is arranged on the surface of the cover disk 64 that faces towards a user 20 in the worn state. The antireflection coating 78 is realized as an interference filter. In principle, however, other implementations of the antireflection coating 78, deemed expedient by someone skilled in the art, would also be conceivable. Furthermore, a color filter may be provided on the further cover disk 64.

The further cover disk 64 is in particular realized as an exchangeable inner protection disk which can be exchanged for different wavelengths.

1. A laser protection device, in particular a laser protection helmet, with at least one optical glare protection filter, which in particular has a nose cutout, with at least one sensor unit which is configured for a detection of a working state and for actuating, as a function thereof, a transmittance of the glare protection filter, with at least one shield unit which is configured to protect a face of a user and in which the at least one glare protection filter is fixedly accommodated, comprising at least one radiation protection unit, which extends around the glare protection filter and at least partially consists of a laser-absorbing, in particular metallic, material.

2. The laser protection device according to claim 1, wherein the at least one radiation protection unit comprises at least one radiation protection wall, which extends around the glare protection filter and consists of a laser-absorbing, in particular metallic, material.

3. The laser protection device according to claim 2, wherein the at least one radiation protection wall consists of aluminum and/or of an aluminum alloy.

4. The laser protection device according to claim 2, wherein the at least one radiation protection wall projects at least substantially perpendicularly to a main extension plane of the glare protection filter into an interior space of the shield unit.

5. The laser protection device according to claim 1, wherein the at least one radiation protection unit comprises at least one shield insert, which consists of a laser-absorbing, in particular metallic, material.

6. The laser protection device according to claim 1, wherein the shield insert is arranged in a mouth region of a user behind the shield unit.

7. The laser protection device according to claim 1, wherein the radiation protection unit is different from the shield unit.

8. The laser protection device according to claim 1, comprising a head fastening unit for a fastening to a head of a user, and an adjustment unit by means of which a position of the at least one shield unit is realized so as to be movable in a defined manner relative to the at least one head fastening unit.

9. The laser protection device according to claim 1, wherein a direct distance between the user's eyes and the optical glare protection filter is realized so as to be adjustable by means of the at least one adjustment unit.

10. The laser protection device according to claim 1, wherein the at least one radiation protection unit comprises at least one face stop, which in an end position of the radiation protection unit is configured to contact the face of a user.

11. A radiation protection unit of a laser protection device according to claim 1.
